(19) United States
(12) Patent Application Publication
(10) Pub. No.: US 2023/0381372 A1
(43) Pub. Date: Nov. 30, 2023

(54) PLA BEADS, DERMAL FILLER INCLUDING THE BEADS, APPARATUS INCLUDING THE FILLER AND USE OF THE APPARATUS

(71) Applicant: Prollenium Medical Technologies, Inc., Richmond Hill (CA)

(72) Inventors: Timothy LEE, Richmond Hill (CA); Shaddi MOGHADAM, Richmond Hill (CA); Khasha IGHANIAN, Richmond Hill (CA); Ario KHOSHBIN, Richmond Hill (CA)

(73) Assignee: Prollenium Medical Technologies, Inc., Richmond Hill, ON (CA)

(21) Appl. No.: 18/197,813

(22) Filed: May 16, 2023

Related U.S. Application Data
(60) Provisional application No. 63/345,742, filed on May 25, 2022.

(51) Int. Cl. A61L 27/18; A61L 27/58

(52) U.S. Cl. CPC A61L 27/18; A61L 27/58; A61L 2400/06; A61L 2430/34

(57) ABSTRACT

Beads for use in dermal filler polymers, which are characterized in that they are spherical, have an average diameter between about 40 microns and about 500 microns and range in diameter between about 20 and about 900 microns. The present invention further includes each of a dermal filler incorporating the beads, an apparatus containing the dermal filler along at least one filler polymer, which is hyaluronic acid or a derivative thereof, and a method for injecting the dermal filler nasolabial skin folds of a patient's face.

### CROSS REFERENCE TO RELATED APPLICATIONS

The present application claims priority from U.S. Ser. No. 63/345,742 filed May 25, 2022.

### FIELD OF THE INVENTION

The present invention relates to the field of dermal fillers.

### BACKGROUND OF THE INVENTION

Dermal fillers based on particulate polylactic acid are known. A well-known filler of this type is that sold under the trademark SCULPTRA by Sanofi. This filler is sold in sealed containers containing, in powder form, poly-L-lactic acid, sodium carboxymethylcellulose and non-pyrogenic mannitol. SCULPTRA filler is hydrated for use.

### SUMMARY OF THE INVENTION

Beads for use in dermal filler polymers form one aspect of the invention. The beads are characterized in that they are spherical, have an average diameter between about 40 microns and about 500 microns and range in diameter between about 20 and about 900 microns.

According to another aspect, the beads can be polylactide based.

According to another aspect, the beads can have an average diameter of about 40 microns and can range in diameter between about 20 microns and about 80 microns.

According to another aspect, the beads can have an average diameter of about 500 microns and can range in diameter between about 400 microns and about 650 microns.

According to another aspect, the beads can be one or more of bioresorbable and biodegradable.

The beads can form part of a dermal filler which forms another aspect of the invention. In addition to the beads, the dermal filler comprises: at least one filler polymer, which is hyaluronic acid or a derivative thereof; and water.

According to another aspect, the filler polymer can be one or more of bioresorbable and biodegradable.

According to another aspect, the filler polymer can contains between about 5 and about 50 mg/ml of the beads.

According to another aspect, the filler polymer can be a round particulate product.

According to another aspect, the filler polymer can be cross-linked.

According to another aspect, the filler polymer can be butanediol diglycidyl ether (BDDE) cross-linked hyaluronan.

According to another aspect, the dermal filler can contain between about 1 and 3 wt % of the filler polymer.

According to another aspect, the dermal filler can contain between about 1 and 4 wt % of the filler polymer.

The dermal filler can form part of an apparatus which defines another aspect of the invention. In addition to the dermal filler, the apparatus comprises a sealed container in which the dermal filler is contained.

According to another aspect, the container can be a syringe.

Forming another aspect of the invention is a use of the apparatus: for injection within the nasobial folds.

According to another aspect, the injection can be one or more of subcutaneous and supraperiosteal.

Advantages, features and characteristics of the invention will become evident upon review of the following detailed description with reference to the appended figures, the latter being briefly described hereinafter.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a graph shown particle size distribution of beads according to Example 1;

FIG. 2 is an optical microscope image of the dermal filler of Example 1;

FIG. 3 is a Mason's trichrome stained slide for 3 implant sites at 52 weeks post implantation of the dermal filler of FIG. 2; and

FIG. 4 is a histomorphic analysis image of FIG. 3; red showing positive collagen stain signals.

### DETAILED DESCRIPTION OF THE PREFERRED EMBODIMENTS

### Beads—Example 1

Ten (10) grams of poly-L-lactic acid having a weight-average molecular weight of about 90,000 was dissolved in 100 ml of methylene chloride to produce a dispersed phase. 10 grams of Polyvinyl alcohol having a weight-average molecular weight of about 100,000 was dissolved in 1000 mL of purified water to produce a continuous phase. The dispersed phased was force through the pores of a membrane wall into a crossflow of the continuous phase to form an emulsion. The membrane had a pore size of 15 um and the flow rates for the dispersed phase and continuous phase were 20 mL/min and 180 mL/min, respectively. The resulting emulsion was stirred gently using an over-head mixer to prevent agglomeration of the droplets until the dichloromethane evaporated from the emulsion and solid PLA microspheres were formed. The resulting microspheres were separated by filtration and dried in a vacuum oven.

Following drying, the spheres were evaluation by laser diffraction using a Malvern Mastersize™ 2000 and were observed to have an average size of 40 um with a very narrow size distribution between about 20 and 80 um, as seen in FIG. 1.

### Example 2

Fifteen (15) grams of poly-L-lactic acid with a weight-average molecular weight of about 90,000 was dissolved in 100 ml of methylene chloride (dispersed phase). 5 grams of Polyvinyl alcohol having a weight-average molecular weight of about 100,000 was dissolved in 1000 mL of purified water (continuous phase). The dispersed phase was forced through the pores of a membrane wall with a pore size of 200 um into a crossflow of the continuous phase to form an emulsion. The flow rate was 20 mL/min for the dispersed phase and 160 mL/min for the continuous phase. The resulting microspheres had a size distribution between about 100 and 1000 um with a majority of the particles between 400-600 um.

### Dermal Filler—Example 3

VLADISAVLJEVIC, G. T. and WILLIAMS, R. A., 2005, Recent Developments in manufacturing emulsions and particular products using membranes. Advances in Colloid and Interface Science, 113(1), incorporated herein by reference, describes bead production processes.

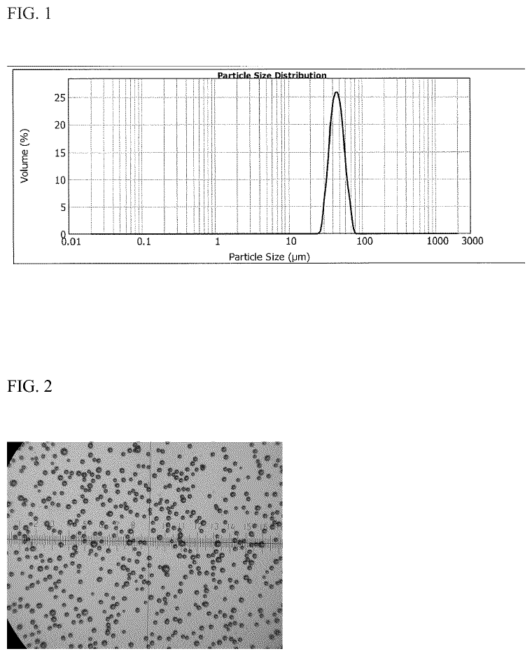

Dermal filler according to an example embodiment of the invention includes hyaluronic acid, beads and water. The beads are as described above in Example 1. The hyaluronic acid and water are of the type commercially available from Prollennium Medical Technololgies, Inc. under the trademark REVANESSE SHAPE. REVANESSE SHAPE will be understood to be hyaluronan cross-linked with butanediol diglycidyl ether (BDDE) to a level that lies above the gel point. The resulting soft solid is milled to fine round gel particles. The gel is purified and pH- and osmolality-balanced by dialysis against filtered phosphate-buffered sale, made from water-for-injection. The dermal filler was produced by combining beads and REVANESSE SHAPE at a ratio of 10 mg:1 g and stirring. FIG. 2 is an optical microscope image of the product; each small division on the photomicrograph represents 14 microns.

Testing

The resulting dermal filler was tested according to ISO 10993 for cytotoxicity, acute, subchronic and chronic toxicity, mutagenecity, sensitization, pyrogenecity, irritation and local effect; all the test results confirmed the product to be safe and biocompatibile.

The local tissue response of the resulting dermal filler at the implantation sites and article degradation was evaluated in comparison to Sculptra™ filler as control, at 2, 13, 26, 52 and 78 weeks after subcutaneous implantation in rats.

A semi-quantitative scoring system according to ISO 10993-6: “tests for local effects after implantation” was used for evaluating the local tissue response of the example and controller fillers. Inflammatory cell infiltrates, necrosis, Neovascularizaton, fibrosis, and fatty infiltration were scored. The values were totaled and then an average score for test and control treatments was calculated.

The average score for the control treatment is subtracted from the test treatment average to determine a reactivity grade based on the scale below:

Reactivity Grade Scale: No/Minimal Reaction (0.0-2.9), Slight Reaction (3.0-8.9), Moderate Reaction (9.0-15.0), Severe Reaction (>15.0)

Table 1 below summarizes the local response results:

TABLE 1

| | Reactivity score | |
|---|---|---|
| Time | Example 1 | Sculptra |
| Week 2 | 10.2 | 13.2 |
| Week 13 | 6.8 | 12.7 |
| Week 26 | 6.5 | 12 |
| Week 52 | 4.9 | 12.4 |
| Week 78 | 5.8 | 11.5 |

At all time points, the example dermal filler showed no tissue reaction compared to the control filler and indeed, at all points in time, mean reactivity for the example dermal filler was lower than that for the control.

A microscopic degradation score of the example dermal filler and control filler was subjectively scored by a pathologist as follows in comparison to the respective baseline implant site at 2, 13, 26, 52 and 78 weeks post-implantation using the scoring set out in Table 2 below.

TABLE 2

| Score | Description |
|---|---|
| 0 | No article found |
| 1 | Article is markedly degraded; some article visible |
| 2 | Article is partially degraded; most article visible |
| 3 | Article appears similar to baseline implantation |
| | with no apparent degradation |

The scoring is set out in Table 3 below:

TABLE 3

| | Degradation | |
|---|---|---|
| | Score | |
| | Test | |
| Time | Filler | Control |
| Week 2 | 3 | 2 |
| Week 13 | 2 | 2 |
| week 26 | 2 | 2 |
| Week 52 | 2 | 2 |
| Week 78 | 1 | 2 |

The implantation endpoints including long-term response (2 weeks, 13 weeks, 26 weeks, 52 weeks) show the example dermal filler to have an in-vivo degradation profile very similar to the control filler.

Collagen is structurally and functionally a key protein of the extracellular matrix. In aged skin collagen fibers fragment, and fibroblasts lose their stability and collapse.

Both the example dermal filler and Sculptra filler stimulate the synthesis and deposition of fibrous tissue and collagen, generating new volume and structural support in a gradual, progressive manner. A semi-quantitative method was used to compare the collagen stimulatory effect of the example dermal filler and the control filler 52 weeks after subcutaneous implantation in rats.

The histomorphometric analysis of Masson's Trichrome (MT) stained slides prepared from each implant sites at 52 week was performed using Image-Pro Plus 7 software to compare the density of collagen at each implant site (for the example filler and control filler). The result is summarized in Table 4 below, wherein:

Positive Collagen Signal Area Density (%): Positive Collagen Signal Area×100 Total ROI Area

TABLE 4

| | | | Positive | Average |
|---|---|---|---|---|
| | | | Collagen | Collagen |
| Time | | Animal | Signal Area | area |
| Point | Group | # | Density | density |
| 52 weeks | Example 1 | 1 | 8.1% | 10.1% |
| | | 2 | 12.7% | |
| | | 3 | 9.6% | |
| | Control | 4 | 8.2% | 8.2% |
| | | 5 | 4.4% | |
| | | 6 | 5.6% | |
| | | 7 | 14.6% | |

The average collagen area density for the example product implant sites were slightly higher than compared to the control.

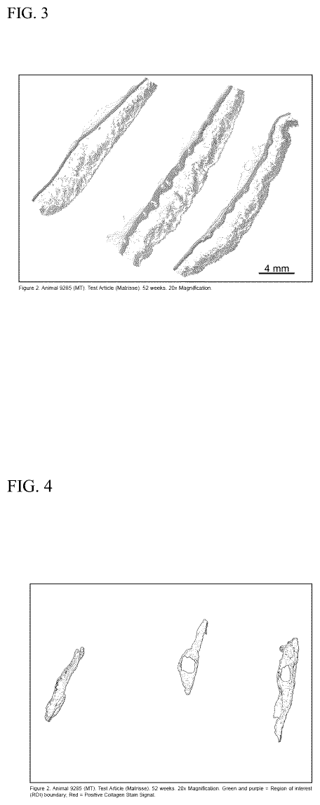

Sample slides of implant sites for the Example 3 filler and control products are shown in FIGS. 3 and 4. FIG. 3 is a Mason's trichrome stained slide for 3 implant sites for the product 52 weeks post-implantation. FIG. 4 is a histomorphic analysis image of the same slide wherein red color shows positive collagen stain signal.

Analysis

The inventive nature of the dermal filler of Example 3 is evidenced upon a review of the following summary Table 5:

TABLE 5

| | Example 3 | Sculptra | Comment |
|---|---|---|---|
| Feature | | | |
| Injection pain | since neutral pH, | varies | Sculptra is often |
| | should be relatively | | reconstituted with |
| | low | | WFI which has a pH |
| | | | of 6.2 |
| Degradation | good | good | |
| Immediate cosmetic | Yes* | No | The cosmetic effect |
| effect | | | of builds over time |
| | | | and then falls as the |
| | | | PLA degrades. [HA |
| | | | based dermal fillers |
| | | | generally have |
| | | | immediate effect*] |
| Mid-term cosmetic | Good* | good | HA based degrade |
| effect | | | slowly in the body; |
| | | | this degradation is |
| | | | offset by the |
| | | | cosmetic effect of |
| | | | the PLA included |
| Long term cosmetic | appears to be as | acceptable | |
| effect | good or better than | | |
| | Sculptra | | |
| Ready-to-use | Yes | No | |

### Example 4

Dermal filler according to another example embodiment of the invention includes hyaluronic acid, beads and water. The beads are as described in Example 2. The hyaluronic acid and water are of the type commercially available from Prollennium Medical Technololgies, Inc. under the trademark REVANESSE ULTRA. REVANESSE ULTRA will be understood to be hyaluronan cross-linked with butanediol diglycidyl ether (BDDE) to a level that lies above the gel point. The resulting soft solid is milled to fine gel particles. The gel is purified and pH- and osmolality-balanced by dialysis against filtered phosphate-buffered sale, made from water-for-injection. The filler was produced by combining the beads and REVANESSE ULTRA at a ratio of 10 mg:1 g and stirring.

This product was tested as indicated above. In this series of tests, the product commercially available from Prollennium Medical Technololgies, Inc. under the trademark REVANESSE CONTOUR XL was used as control. The reactivity and degradation tests at 4 and 13 weeks are shown in Tables 6 and 7, respectively.

TABLE 6

| | Reactivity score | |
|---|---|---|
| Time | Example 2 | Control |
| Week 4 | 5.2 | 5 |
| Week 13 | 5 | 5 |

TABLE 7

| | Degradation | |
|---|---|---|
| | score | |
| Time | Example 2 | Control |
| Week 4 | 2 | 2 |
| Week 13 | 2 | 2 |

Analysis

From the results available thus far, Example 4 also appears to be useful in that both reactivity and degradation scores were acceptable.

### Apparatus

### Example 5

Apparatus according to an example embodiment of the invention comprises a container and a dermal filler composition. The container is a 1 ml sealed syringe. The dermal filler composition is interior of the syringe and comprises 1 ml of the filler of Example 3.

Testing

A plurality of the syringes were held at 5° C. for a 78 week period; a further plurality were held at 25° C. for a 78 week period. The contents of the syringes were examined at weeks 4, 12, 26, 39, 52 and 78, over the period for compliance with the following standard:

| USP Sterility | Sterile | |
|---|---|---|
| Endotoxin (LAL) | <0.5 | EU/mL |
| HA Concentration | 22-28 mg/g | |
| Residual BDDE | <2 | ppm |
| PLA content | 7-13 mg/g | |
| Osmolality | 260-360 mOsm/kg | |
| Extrusion Force | ≤5.0 | lbs |

| | |
|---|---|
| pH | 6.8-7.6 |

All the stability indicating parameters were within the acceptable ranges during the evaluation period.

Use:

Forming yet another aspect of the invention is a corresponding method for utilizing the inventive apparatus for injection within the nasobial folds. The injection can be one or more of subcutaneous and supraperiosteal.

Variations

Whereas specific embodiments are herein shown and described, it will be evident that variations are possible.

Without Limitation in this Regard:
whereas a specific polylactide polymer is mentioned, other polylactide polymers can be used and indeed, the beads could be formed of other known biocompatibles such as PLGA, PLA and polycaprolactam;
whereas specific sizes of beads are indicated, others could be used;
whereas two types of hyaluronic acid have been used, other hyaluronic acid, including non-cross-linked versions, would be expected to be useful;
whereas in the examples, about 10 mg/ml of particles are used, US20220133951 teaches the utility of up to about 50 mg/ml of particles, as such, utility of a range between 0 and 50 can be reasonable predicted to have utility;
whereas in the examples, dermal fillers containing 2.5 wt % HA are used, the use of dermal fillers between containing between 1 and 4 wt % HA is commonplace in the industry and it is reasonable to predict utility throughout that range; and
whereas a specific size syringe is mentioned, other sizes could be used.

Accordingly, the invention should be understood to be limited only by the accompanying claims, purposively construed.

Having described my invention, other and additional preferred embodiments will become apparent to those skilled in the art to which it pertains, and without deviating from the scope of the appended claims. The detailed description and drawings are further understood to be supportive of the disclosure, the scope of which being defined by the claims. While some of the best modes and other embodiments for carrying out the claimed teachings have been described in detail, various alternative designs and embodiments exist for practicing the disclosure defined in the appended claims.

The foregoing disclosure is further understood as not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, a person of ordinary skill in the art will recognize that changes may be made in form and detail without departing from the scope of the present disclosure. Thus, the present disclosure is limited only by the claims.

In the foregoing specification, the disclosure has been described with reference to specific embodiments. However, as one skilled in the art will appreciate, various embodiments disclosed herein can be modified or otherwise implemented in various other ways without departing from the spirit and scope of the disclosure. Accordingly, this description is to be considered as illustrative and is for the purpose of teaching those skilled in the art the manner of making and using various embodiments of the disclosure. It is to be understood that the forms of disclosure herein shown and described are to be taken as representative embodiments. Equivalent elements, materials, processes or steps may be substituted for those representatively illustrated and described herein. Moreover, certain features of the disclosure may be utilized independently of the use of other features, all as would be apparent to one skilled in the art after having the benefit of this description of the disclosure. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

Further, various embodiments disclosed herein are to be taken in the illustrative and explanatory sense, and should in no way be construed as limiting of the present disclosure. All joinder references (e.g., attached, affixed, coupled, connected, and the like) are only used to aid the reader's understanding of the present disclosure, and may not create limitations, particularly as to the position, orientation, or use of the systems and/or methods disclosed herein. Therefore, joinder references, if any, are to be construed broadly. Moreover, such joinder references do not necessarily infer that two elements are directly connected to each other.

Additionally, all numerical terms, such as, but not limited to, “first”, “second”, “third”, “primary”, “secondary”, “main” or any other ordinary and/or numerical terms, should also be taken only as identifiers, to assist the reader's understanding of the various elements, embodiments, variations and/or modifications of the present disclosure, and may not create any limitations, particularly as to the order, or preference, of any element, embodiment, variation and/or modification relative to, or over, another element, embodiment, variation and/or modification.

It will also be appreciated that one or more of the elements depicted in the drawings/figures can also be implemented in a more separated or integrated manner, or even removed or rendered as inoperable in certain cases, as is useful in accordance with a particular application. Additionally, any signal hatches in the drawings/figures should be considered only as exemplary, and not limiting, unless otherwise specifically specified.

1. Beads for use in dermal filler polymers, the beads being characterized in that they are spherical, have an average diameter between about 40 microns and about 500 microns and range in diameter between about 20 and about 900 microns.

2. Beads according to claim 1, further characterized in that they are polylactide based.

3. Beads according to claim 1, further characterized in that they have an average diameter of about 40 microns and can range in diameter between about 20 microns and about 80 microns.

4. Beads according to claim 1, further characterized in that they have an average diameter of about 500 microns and can range in diameter between about 400 microns and about 650 microns.

5. Beads according to claim 1, further characterized in that they are one or more of bioresorbable and biodegradable.

6. A dermal filler comprising:
beads characterized in that they are spherical, have an average diameter between about 40 microns and about 500 microns and range in diameter between about 20 and about 900 microns;
at least one filler polymer, which is hyaluronic acid or a derivative thereof; and
water.

7. Dermal filler according to claim 6, wherein the filler polymer is one or more of bioresorbable and biodegradable.

8. Dermal filler according to claim 6, characterized in that it contains between about 5 and about 50 mg/ml of the beads.

9. Dermal filler according to claim 6, wherein the filler polymer is a particulate product, the particles being round.

10. Dermal filler according to claim 6, wherein the filler polymer is cross-linked.

11. Dermal filler according to claim 10, wherein the filler polymer is butanediol diglycidyl ether (BDDE) cross-linked hyaluronan.

12. Dermal filler according to claim 6, characterized in that it contains between about 1 and 3 wt % of the filler polymer.

13. Dermal filler according to claim 6, characterized in that it contains between about 1 and 4 wt % of the filler polymer.

14. An apparatus comprising:
a sealed container; and
interiorly of the sealed container, a dermal filler having beads characterized in that they are spherical, have an average diameter between about 40 microns and about 500 microns and range in diameter between about 20 and about 900 microns;
at least one filler polymer, which is hyaluronic acid or a derivative thereof; and
water.

15. Apparatus according to claim 14, wherein the container is a syringe.

16. A method for injecting a dermal filler, comprising:
providing a sealed container containing a dermal filler having beads characterized in that they are spherical, have an average diameter between about 40 microns and about 500 microns and range in diameter between about 20 and about 900 microns;
combining into the dermal filler at least one filler polymer including at least one of a hyaluronic acid or derivative thereof; and
injecting with a syringe the filler and polymer into nasolabial skin folds of a patient's face.

17. The method as described in claim 16, further comprising the step of injecting the syringe in one of subcutaneous and supraperiosteal locations.
